(19) United States
(12) Patent Application Publication
(10) Pub. No.: US 2015/0118695 A1
(43) Pub. Date: Apr. 30, 2015

(54) PORTABLE POCKET-SIZED BIOSENSOR

(71) Applicant: Korea University Research and Business Foundation, Seoul (KR)

(72) Inventor: Sehwan Paek, Seoul (KR)

(73) Assignee: Korea University Research and Business Foundation

(21) Appl. No.: 14/516,149

(22) Filed: Oct. 16, 2014

(30) Foreign Application Priority Data
Oct. 28, 2013 (KR) 10-2013-0128329

(51) Int. Cl. G01N 33/543; G01N 21/64; G01N 33/569

(52) U.S. Cl. CPC G01N 33/54373; G01N 33/56916; G01N 21/6428; G01N 2021/6439; G01N 2201/067
USPC 435/7.92; 422/421; 435/287.2; 436/501; 435/7.1

(57) ABSTRACT

Disclosed herein is a portable biosensor, wherein a fluidic channel, comprising a porous solid matrix on which a capture recognition material of an analyte is fixed, is in contact with a signal detection sensor, wherein the signal detection sensor is a lens-free complementary metal oxide-semiconductor (CMOS) image sensors (CISs) for measuring a light signal generated by the reaction between the analyte and the capture recognition material.

### CROSS REFERENCE TO RELATED APPLICATION

This application claims the benefit of Korean Patent Application No. 10-2013-0128329, filed Oct. 28, 2013, which is hereby incorporated by reference in its entirety into this application.

### BACKGROUND OF THE INVENTION

1. Technical Field

The present invention relates generally to a portable biosensor and, more particularly, to a portable pocket-sized biosensor arranged by locating a lens-free complementary metal-oxide-semiconductor (CMOS) image sensors (CISs) as a signal detection device in the proximity of a light signal-generating part to save space required for a camera's focus thereby enabling its miniaturization.

2. Description of the Related Art

An immunoassay is an analytical method based on antigen-antibody binding reactions, and for more than half a century has been used for the purpose of quantifying numerous organic materials as analytes. Initially, the antigen-antibody binding reactions were performed in a liquid phase but can now be performed on solid matrices. The transition was made possible by the distribution of 96-well microtiter plates to the public along with numerous attempts via analytical experiments to attain an easier separation of antigen-antibody binding complexes.

In fact, many signal generators have been used to trace the antigen-antibody binding complexes, but enzymes in particular have been used due to their ease of application. The enzyme-based immunoassay called enzyme-linked immunosorbent assay (ELISA) enables the handling of multiple samples simultaneously. However, the method has disadvantages in that it is a step-by-step reaction procedure requiring separation of unused reagents at each step, and also requires labor, expertise, and laboratory facilities for signal measurements. Accordingly, as an alternative, immunochromatographic analysis has been introduced, which with its rapid one-step assay, enables performing analysis on a porous membrane as a solid matrix in combination with colloidal gold as a tracer. Recently, a fluidic channel-based assay has been also available in this category.

Additionally, a novel format of immunoassay has been developed as a point-of-care technology based on ELISA, i.e., ELISA-on-a-chip (EOC), which can provide an analysis capable of detecting low analyte concentrations and also can be utilized under non-laboratory conditions. However, the EOC in current use has problems in that it requires a camera-type detector for measuring color or light signals generated during the assay and this kind of detector is too bulky to be carried by hand.

### SUMMARY OF THE INVENTION

Accordingly, the present invention has been made keeping in mind the above problems occurring in the prior art, and an object of the present invention is to provide a portable pocket-sized biosensor by locating a lens-free complementary metal oxide-semiconductor (CMOS) mage sensors (CISs) at a position where a signal is generated by a reaction between an analyte and a recognition material in a fluidic channel.

In order to accomplish the above object, the present invention provides a portable biosensor, comprising a fluidic channel, wherein the fluidic channel comprises a porous solid matrix on which a capture recognition material of an analyte is fixed, and is in contact with a signal detection sensor, said signal detection sensor being a lens-free CIS for measuring a light signal generated by the reaction between the analyte and the capture recognition material.

Also, the present invention provides a method for detecting an analyte, including:

a) loading an analyte into a sample application port of a fluidic channel comprising a porous solid matrix, to which a capture recognition material of an analyte is fixed;

b) generating a light signal by a reaction between the analyte and the capture recognition material in the fluidic channel; and

c) detecting the analyte by measuring the light signal with the portable biosensor described above.

### BRIEF DESCRIPTION OF THE DRAWINGS

The above and other objects, features and advantages of the present invention will be more clearly understood from the following detailed description taken in conjunction with the accompanying drawings, in which:

FIGS. 1A to 1C provide pictures illustrating the measuring steps for a portable biosensor of the present invention, where FIG. 1A is a picture showing the vertical flow of a sample supplied to a fluidic channel, and a schematic diagram showing a sample, a capture antibody, and an enzyme-labeled detection antibody; and FIG. 1B is a picture showing the horizontal flow of an enzyme substrate solution, and a schematic diagram showing the generation of a light signal by the reaction between an enzyme and a substrate; and FIG. 1C is a picture of a measuring device for measuring the light signal generated;

FIG. 2A is a schematic diagram showing a structure where a lens-free CIS is assembled in direct contact with a fluidic channel; FIG. 2B is a schematic diagram showing a structure where a CIS is disposed on top of a PCB; and FIG. 2C is a picture showing images of a control signal and an analyte signal measured by a CIS;

FIG. 3 is a picture showing a biosensor for measuring a light signal under dark conditions;

FIG. 4A is a graph showing light signals according to the HRP enzyme concentration in standard samples; and FIG. 4B is a graph showing the result of color development according to the HRP enzyme concentration;

FIG. 5 is an image of chemiluminometric signals or colorimetric signals according to Salmonella concentration. In particular, (A) is an image of chemiluminometric signals according to Salmonella concentration measured by CIS; (B) is an image of chemiluminometric signals according to Salmonella concentration measured by cooled-CCD; and (C) is an image of colorimetric signals measured according to Salmonella concentration;

FIG. 6A is a graph showing dose-responses of Salmonella measured by a CIS; and FIG. 6B is a graph showing chemiluminometric signals measured by a CIS or cooled-CCD as signal detection sensor and digitized colorimetric signals measured by a web camera according to Salmonella concentration;

FIG. 7A is a picture of a portable biosensor combined with a magnetic enrichment module; FIG. 7B is a picture showing concentrating bacteria via magnetic separation; and FIG. 7C is a picture showing an elution process of bacteria concentrated on the surface of the magnetic beads;

FIG. 8A is a graph showing chemiluminometric signals according to Salmonella concentration with magnetic enrichment or without the enrichment of the analyte; and FIG. 8B is a graph showing determination of the magnetic enrichment yield after converting the chemiluminometric signals of an analyte according to Salmonella concentration when a magnetic enrichment was not performed on the analyte via logit-log transformation; and

FIG. 9 is a graph showing determination of pre-cultivation times of Salmonella (inoculated in 3.3 CFU/10 g) with magnetic enrichment or without the enrichment.

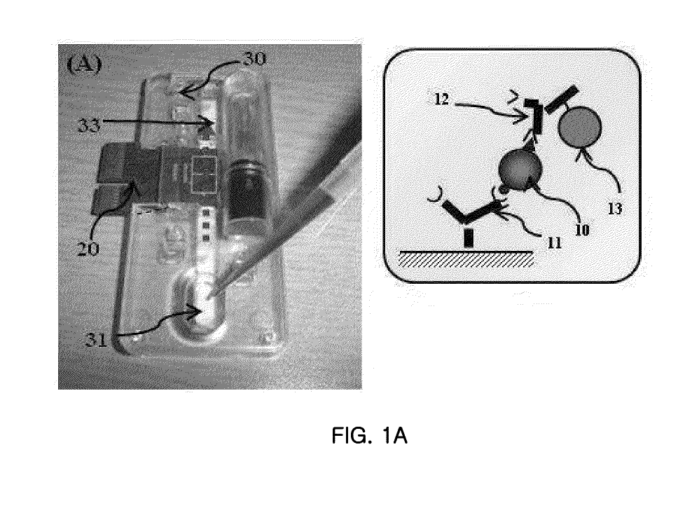

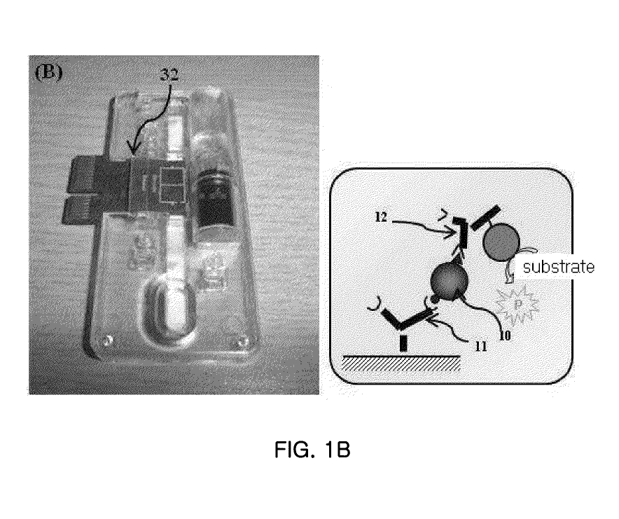

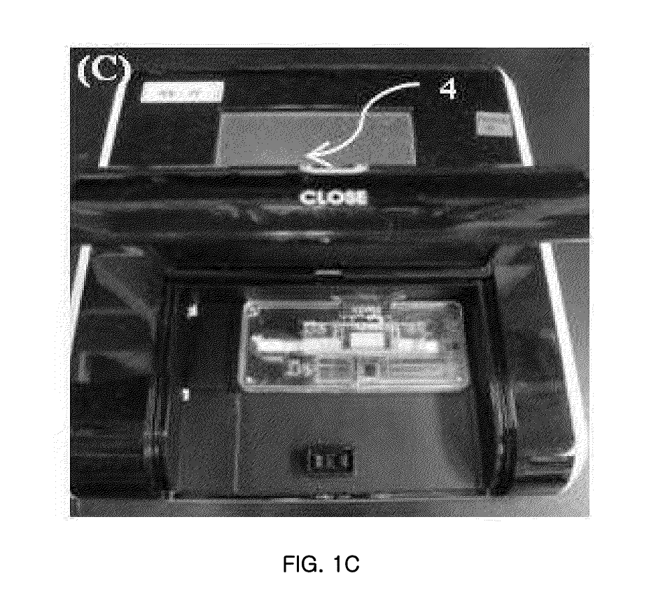

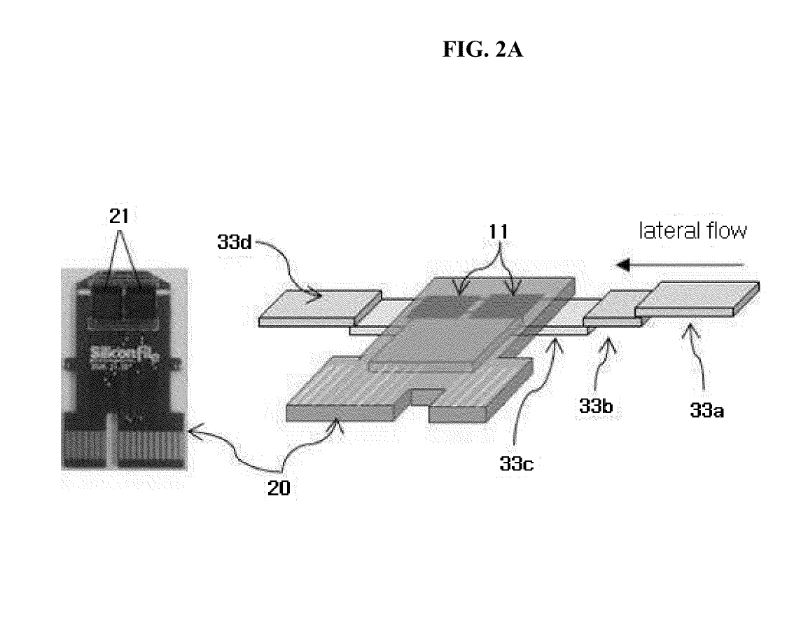

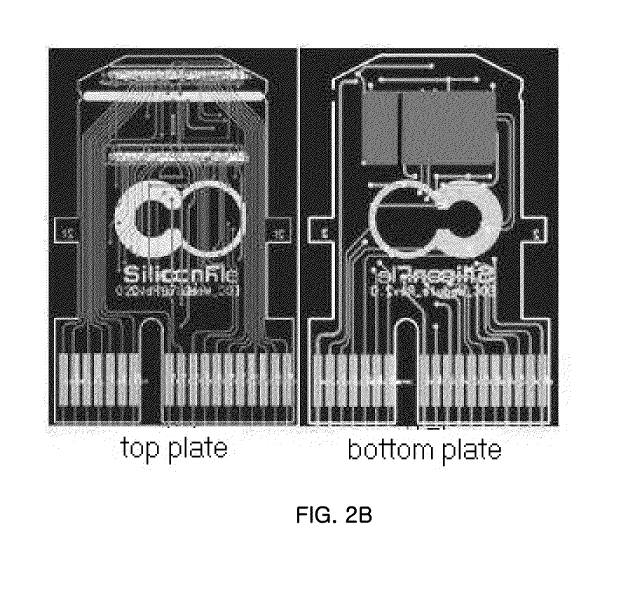

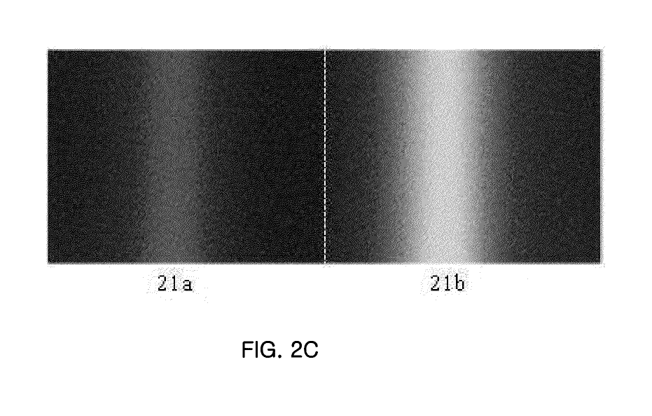

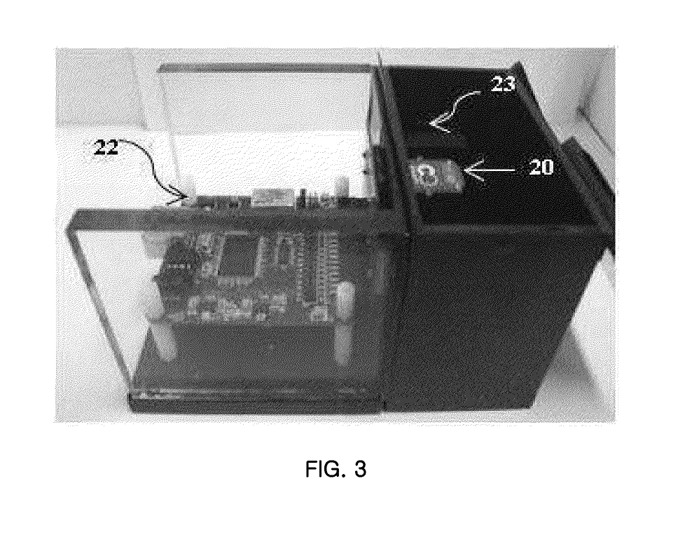

### DESCRIPTION OF THE PREFERRED EMBODIMENTS

Below, a detailed description will be given of the present invention.

The present invention addresses a portable biosensor comprising a fluidic channel, wherein the fluidic channel includes a porous solid matrix on which a capture recognition material of an analyte is fixed, and is in contact with a signal detection sensor, said signal detection sensor being a lens-free complementary metal oxide-semiconductor (CMOS) image sensors (CISs) for measuring a light signal generated by the reaction between the analyte and the capture recognition material.

The portable biosensor of the present invention enables an efficient photon delivery by placing a light detection sensor in the proximity of a light signal-generating area in the fluidic channel, and it can be miniaturized by saving space for a focal distance required by a camera using a lens-free CIS. Accordingly, the portable biosensor of the present invention can be manufactured into a pocket-sized device, and thus can be used for point-of-care diagnosis of diseases in non-laboratory settings, e.g., hygiene monitoring in cafeterias, measurement of toxins, agricultural chemicals, harmful substances, and food additives present in foods or drinking water in non-laboratory settings.

The fluidic channel is an immunochromatographic device based on a cross flow in the form of EOC, an immunoassay platform based on a cellulose membrane and is a portable device enabling a fast immunoassay. EOC employs a method for measuring antigens or antibodies via an antigen-antibody reaction using an enzyme as a marker. EOC can perform a sensitive analysis for analytes at low concentrations and also reduce time required for analysis by allowing the analytes to cross the fluidic channel vertically and horizontally.

The porous solid matrix to be used may be at least one selected from the group consisting of glass fiber membrane, nitrocellulose (NC) membrane, cellulose membrane, nylon membrane, and polyvinylidene difluoride membrane.

Additionally, the capture recognition material being fixed onto the fluidic channel is at least one selected from the group consisting of an antibody reacting with an analyte or the conjugated analyte, an enzyme, a receptor, hexane, an aptamer, a peptide, and a molecular-imprinted artificial membrane.

The fluidic channel consists of four different kinds of membrane pads. The fluidic channel is arranged in the order of a sample application pad 33a, a conjugate pad 33b, a signal generation pad 33c and a sample absorption pad 33d based on a sample application port 31, and the neighboring pads are partially overlapped. A recognition material capable of detecting an analyte is fixed on the conjugate pad 33b. Additionally, analytical lines 21b and control lines 21a are provided on the signal generation pad 33c. Besides, a recognition material for capturing an analyte is fixed on the analytical lines 21b whereas a second recognition material for an enzyme-labeled detection antibody is fixed on the control lines 21a, respectively, thereby enabling a generation of a constant light signal on the control lines 21a regardless of the concentration of the analyte.

A dried enzyme-labeled detection antibody is used on the conjugate pad 33b. When the enzyme-labeled detection antibody is used, the enzyme acts as a signal generating source by reacting with a substrate solution, thereby improving analytical performance.

CIS of the present invention used as a signal detection sensor was fixed by mounting it on a printed circuit board (PCB), and was respectively disposed on the analytical lines 21b and the control lines 21a of the signal generation pad 33c, which generates light signals, thereby allowing it to measure light signals.

The light signal is luminescent, fluorescent, and colorimetric, and the signal detection sensor used for measuring the light signal may include a lens-free charge coupled device (CCD), a photodiode, or a photomultiplier tube in addition to CIS.

The portable biosensor of the present invention having a structure as described above may detect materials by a method including:
a) loading an analyte into a sample application port 31 of a fluidic channel including a porous solid matrix, to which a capture recognition material of an analyte is fixed;
b) generating a light signal by a reaction between the analyte and the capture recognition material in the fluidic channel; and
c) detecting the analyte by measuring the light signal with the portable biosensor described above.

More specifically, when an analyte is loaded into the sample application port 31 of the fluidic channel, the analyte is flown in a vertical direction relative to the fluidic channel, and the analyte is reacted with a recognition material which is bound to the dried enzyme on the conjugate pad 33b of the fluidic channel. Then, the reacted analyte is introduced onto the signal generation pad 33c, and captured by a capture recognition material fixed on the signal generation pad 33c. Here, an enzyme substrate solution is introduced onto the signal generation pad 33c in a horizontal direction to be crossed, and the substrate solution reacts with the enzyme conjugated to the recognition material and generates a light signal, which is measured via CIS in the dark. The measured light signal is converted into an electric signal by a signal detection device thereby capable of measuring analytes.

The signal detection device for converting a light signal into an electric signal is a device where an analog-to-digital converter, a central processing unit, a synchronous random access memory, a volatile memory (NAND or NOR), a secure digital card, and a liquid crystal display unit are assembled thereinto.

Additionally, the sensitivity of the portable biosensor of the present invention may be improved by applying a magnetic enrichment technology using magnetic beads on which capture recognition materials of an analyte are fixed prior to loading the analyte into the sample application port 31. Magnetic beads consist of a compartment for separation where bacteria are magnetically separated within the magnetic field, a medium absorption compartment for absorbing a water-containing medium using a membrane pad, and a compartment for analysis for performing immunoassay using the portable biosensor of the present invention.

After placing a magnet 56 on top of a separation tubing 55, the immuno-magnetic beads combined with recognition materials were added into a sample mixed with bacteria to react, and the solution was added into the sample inlet port 50. Here, the solution moves along the separation tubing 55 into the medium absorption pad 53 regulated by a 3-way stopcock 51, and the magnetic bead-bound bacteria present in the solution retained within the separation tubing due to the magnet 56. The magnetic bead-bound bacterial cells within the separation tubing 55 are dissociated by adding a small amount of an acidic solution, and the thus dissociated bacterial cells are transferred into the sample application port 31 of the portable biosensor located in the compartment for analysis by adjusting the 3-way stopcock 51. Here, the concentrated bacterial sample is neutralized to a pH suitable for immunoassay by reacting with an alkaline reagent accumulated after drying on the sample application pad 33a, and the analyte is subjected to analysis in the same manner as in the analytical procedure for the portable biosensor described above.

The present invention will be described in detail below with reference to the accompanying drawings. Repeated descriptions and descriptions of known functions and configurations which have been deemed to make the gist of the present invention unnecessarily obscure will be omitted below. The embodiments of the present invention are intended to fully describe the present invention to a person having ordinary knowledge in the art to which the present invention pertains. Accordingly, the shapes, sizes, etc., of components in the drawings may be exaggerated to make the description clearer. Furthermore, exemplary embodiments regarding the measurement of Salmonella are provided herein below.

### Manufacture of a Portable Biosensor and Detection of Salmonella Using the Same

### Example 1

### Manufacture of a Portable Biosensor

### (1) Binding of Recognition Material (Enzyme and Detection Antibody)

1-1. A Crosslinker Method

Anti-Salmonella detection antibodies (0.5 mg/mL, 100 mL) were dissolved in a mixed solution containing 100 mM phosphate buffer solution (pH 7.4), 140 mM sodium chloride (100 mM phosphate buffered saline, PBS), 10 mM DTT (dithiotheritol, Pierce) and 5 mM EDTA, and reacted for 1 hour at 37° C. to activate sulfhydryl group (—SH). An excess reagent was removed via Sephadex G-15 gel column

In order to provide maleimide group by activating horseradish peroxidase (HRP; 25 mg/mL, 56.8 mL), an enzyme used in this Example, HRP was reacted with a 30 mole ratio of succinimidyl-4-(N-maleimidomethyl)cyclohexane-1-carboxylate (SMCC, Pierce) dissolved in 100 mM PBS for 2 hours at room temperature, and an excess reagent was removed via gel column. The activated anti-Salmonella detection antibodies were reacted in 10 mole excess ratio with the activated HRP for 2 hours at room temperature and conjugated. The conjugated complex of HRP-anti-Salmonella detection antibodies was mixed with an equal volume of glycerol and stored at 4° C.

1-2. Method of Direct Conjugation

HRP was activated by dissolving HRP (5 mg/mL) in 100 mM acetated buffer solution (pH 5.5) followed by reacting with sodium periodate (final 10 mM) for 20 minutes at room temperature. The replacement of an excess reagent and a buffer solution was performed via G-15 column using 100 mM carbonate buffer solution (pH 9.5). The activated HRP was reacted in 5 mole ratio with anti-Salmonella detection antibodies for 3 hours at 4° C., and then reacted further with sodium borohydrate (final 2 mM) for 15 minutes. Immediately thereafter, the conjugated complex of HRP-anti-Salmonella detection antibodies was neutralized with 1 M phosphate buffer solution (pH 7.0), dialyzed in 10 mM PBS, and stored in the presence of a 50% glycerol at 4° C.

### (2) Preparation of EOC

Four different kinds of membrane pads were aligned in the order of the sample application pad 33a (glass membrane; 4×17 mm; Grade 319), the conjugate pad 33b (glass membrane; 4×10 mm; PT-R5), the signal generation pad 33c (NC membrane; 4×25 mm; 90CNPH-N-SS40), and the sample absorption pad 33d (cellulose membrane; 4×15 mm; 17CHR) relative to the sample application port 31. The adjacent pads were aligned each other to be partially overlapped.

The sample application pad 33a was prepared by allowing it to absorb a mixed solution containing 1% casein-PBS and 0.5% Tween-20 (30 mL/pad) followed by drying at 60° C. for 1 hour.

The conjugate pad 33b was prepared by allowing it to absorb a mixed solution containing 100 mM PBS (pH 7.4), 5% casein, 5 mM ascorbic acid, 0.5% Tween-20 and 20% trehalose (13.8 mL), and further with the HRP-anti-Salmonella detection antibody conjugate prepared in Example 1 (0.2 mL; final 0.1 mg/mL).

The signal generation pad 33c for generating signals was coated with anti-Salmonella capture antibodies (100 mM PBS containing 3% trehalose (1 mg/mL)) on the analytical lines 21b, and with goat anti-rabbit IgG (0.05 mg/mL) on the control lines 21a, 6 mm and 11 mm distant from the bottom, respectively, using a microsyringe at a rate of 1.5 mL/cm (BioJet3000, BioDot, Irvine, Calif., USA). The signal generation pad 33c was dried at 37° C. for 1 hour, and stored in a plastic bag along with a desiccant.

The four different kinds of pads described above were assembled on plastic films in the form of a 4 mm wide membrane strip using a double-sided tape. The reverse side of the signal generation pad 33c was pasted with a black tape to prevent mutual interference between light signals.

### (3) Manufacture of CIS Module

A disposable module to be used as a signal detection sensor for measuring the light signal generated in the fluidic channel of EOC was manufactured by placing two CISs (3.0×3.0 mm, 1.3 megapixel array) on a printed circuit board (PCB).

A CIS module consists of three functional parts; a sensing part, a current circulation part, and a part for transporting a signal to a detection device. Two CISs were respectively aligned on the analytical lines 21b and the control lines 21a on the signal generation pad 33c when the module and EOC were assembled.

The electric wiring was densely distributed between CIS chipset and the output terminal pad according to the design to accomplish its integration in a small space. The electric wire patterning of PCB was performed using gold plating, and stably fixed on the board. The circuit connecting the apertures for binding wiring layers on top and bottom of the PCB was coated with silk, a non-conductive material to stably increase CIS performance. The electric wires extended from the chipset were coated with epoxy to protect them in an aqueous environment.

### (4) Integration of CIS with EOC

The EOC plastic cartridge (32×76×8 mm) consisted of two plates; the top plate and the bottom plate. It was configured such that the fluidic channel (EOC) can be mounted on the vertical fluidic channel when the plates were assembled, and the substrate solution from its tank can flow across the signal generation pad 33c in the horizontal fluidic channel to be absorbed onto the horizontal flow absorption pad. The top plate included a panel for monitoring signals and an inlet port for introducing samples. Additionally, the top plate was designed with a pattern using an etching machine controlled by a computer so that a CIS module can be mounted in the proximity of the signal generation pad 33c. The thus manufactured portable EOC-CIS integrated biosensor was stored at room temperature with a desiccant.

### (5) Assembly of a Signal Detection Device

A signal detection device which can be connected to a CIS module was manufactured to convert a light signal to an electric signal.

An analog-to-digital converter, a central processing unit, a synchronous random access memory, two volatile memories (NAND or NOR), a secure digital card, and a liquid crystal display unit were assembled into a single device. The photons received by CIS during a signal formation were converted into electrons by photodiodes, and then converted to voltage by an amplifier. The voltage was converted to a digital data via an analog-to-digital converter, which was then used in mathematical calculation using the central processing unit of a personal computer. The processed data were stored temporarily in the synchronous random access memory, and then in an external secure digital card along with a light image produced thereof. The measured signal was converted to a concentration of an analyte by using the standard curve installed in an additional memory by a firmware of the central processing unit. The selection of an execution command may be controlled by a touch input device on the uppermost panel of the detection device.

### Experimental Example 1

### Confirmation of Analytical Performance of CIS

In order to confirm the analytical performance of CIS, a sample for analysis was directly added onto CIS and confirmed the analytical performance and detection limit of CIS.

HRP was diluted in casein-PBS solution at varied concentrations (0, 10, 50, 100 fmol/mL, 1, and 10 pmol/mL), and 1 μL each of the diluted HRP was loaded onto a CIS module connected to a detection device. Immediately thereafter, 1 mL of chemiluminometric substrate solution was added thereto, and the light signals generated therefrom according to each of the varied HRP concentrations were measured in the dark. The signal images captured about two minutes thereafter were stored in a personal computer and confirmed the results.

Additionally, the signal colors according to the varied HRP concentrations were observed in a microtiter plate using a colorimetric soluble TMB (200 μL). The reaction was performed for 15 minutes. After adding 2 M sulfuric acid solution (50 μL), the color signals were measured at the absorbance of 450 nm

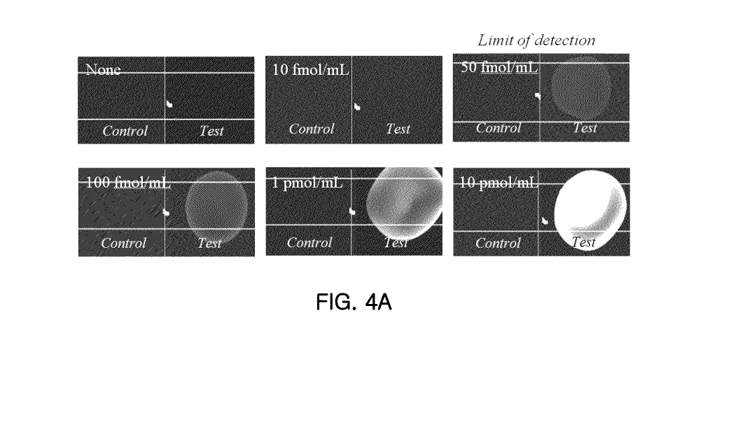

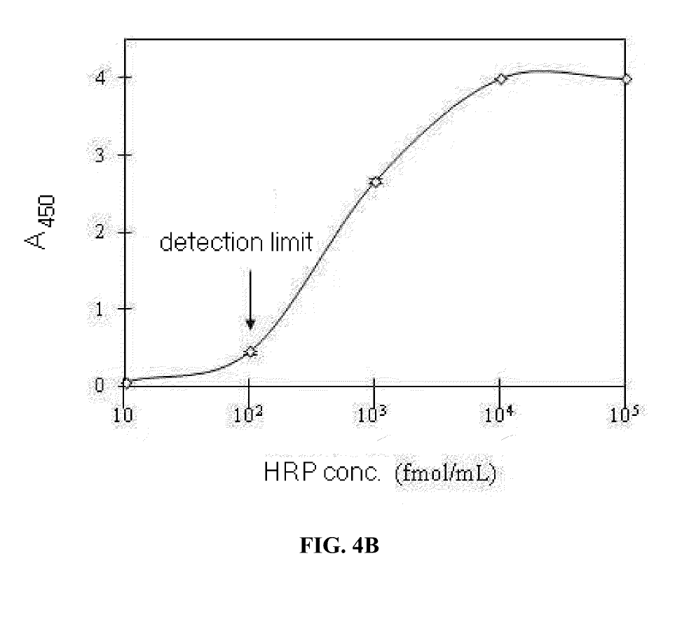

Based on the images captured by CIS, it was confirmed that the light signal was increased proportionally to the enzyme (HRP) concentration (FIG. 4A), and the detection limit of the enzyme was 50 fmol/mL. The light signal was generated on the analytical lines 21b but not on the control lines 21a without the enzyme.

Additionally, the assay on a microtiter plate using TMB also showed that the colorimetric signal increased proportionally according to the enzyme concentration (FIG. 4B), thus confirming that the light signal measurement using CIS is highly reliable.

### Experimental Example 2

### Detection of Salmonella According to Types of Signal Detection Sensors (CIS, Cooled-CCD, and Web Camera)

Samples containing varied concentrations of Salmonella (Salmonella typhimurium (S. typhimurium); KCCM 11806) in the amount of 100 μL, respectively, were loaded into the fluidic channel (EOC), and the vertical flow was allowed for 15 minutes thereby completing immune responses. The horizontal absorption pad for the horizontal flow was attached next to the signal-generating signal generation pad 33c to be partially overlapped, and supplied with a substrate solution (150 mL) containing a chemiluminometric substrate.

The signal intensity according to Salmonella concentration was measured using signal detection sensors such as CIS and cooled-CCD, and the captured images were quantified via optical density and then used to prepare a dose-response curve.

Additionally, an immunoassay on Salmonella was performed by measuring the colorimetric signals. Samples containing Salmonella serially diluted with casein-PBS-Tween-20 (100 μL) were prepared, and a vertical flow was processed for 15 minutes by loading the samples into the fluidic channel (EOC). The horizontal flow absorption pad was disposed next to the signal generation pad 33c to be partially overlapped, and an HRP substrate containing TMB-M (150 μL) was loaded into the substrate inlet port. After horizontal flow for 10 minutes, when the colorimetric signal occurred on the signal generation pad 33c, the images were captured using a web camera, and quantified along the central line using computer software.

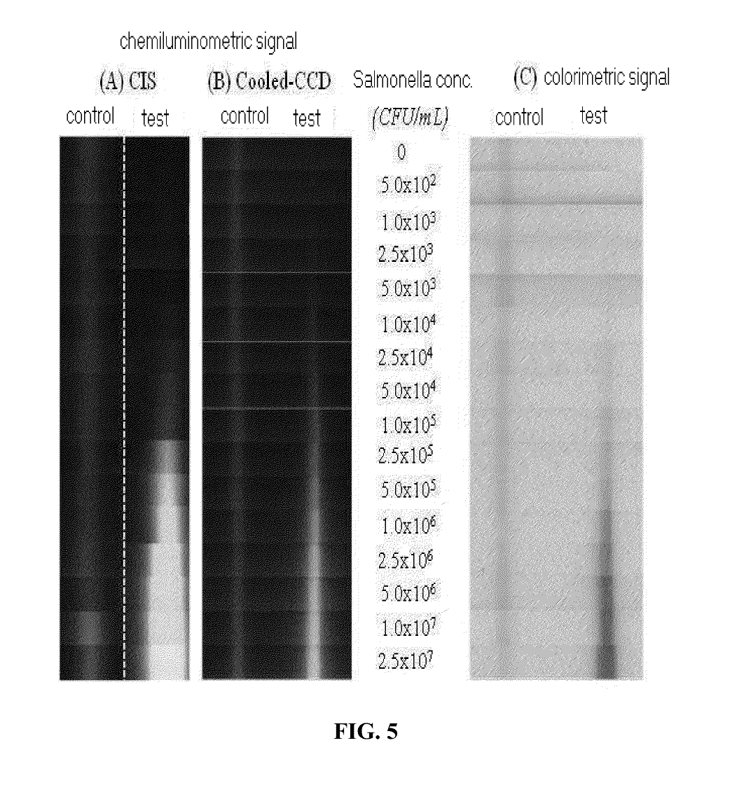

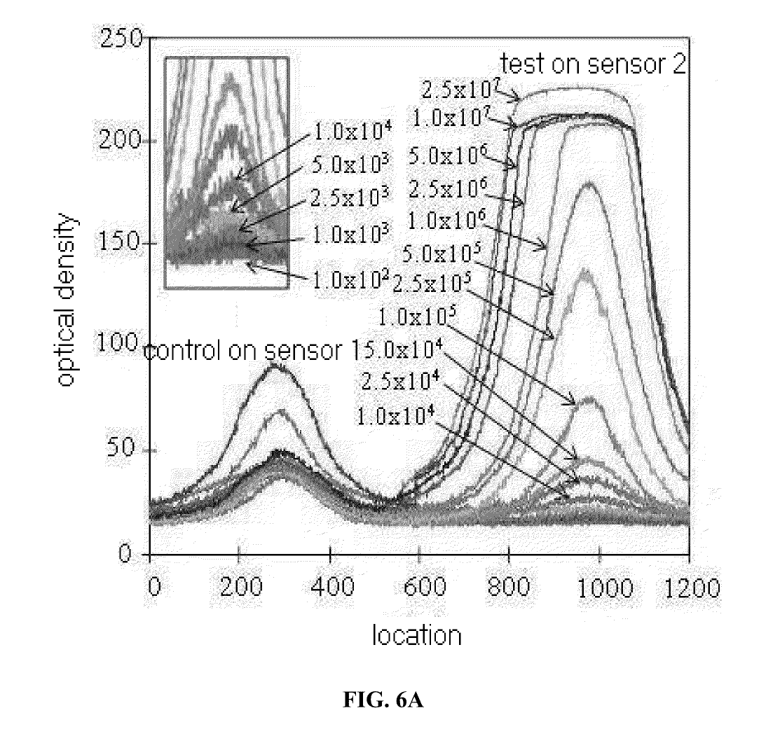

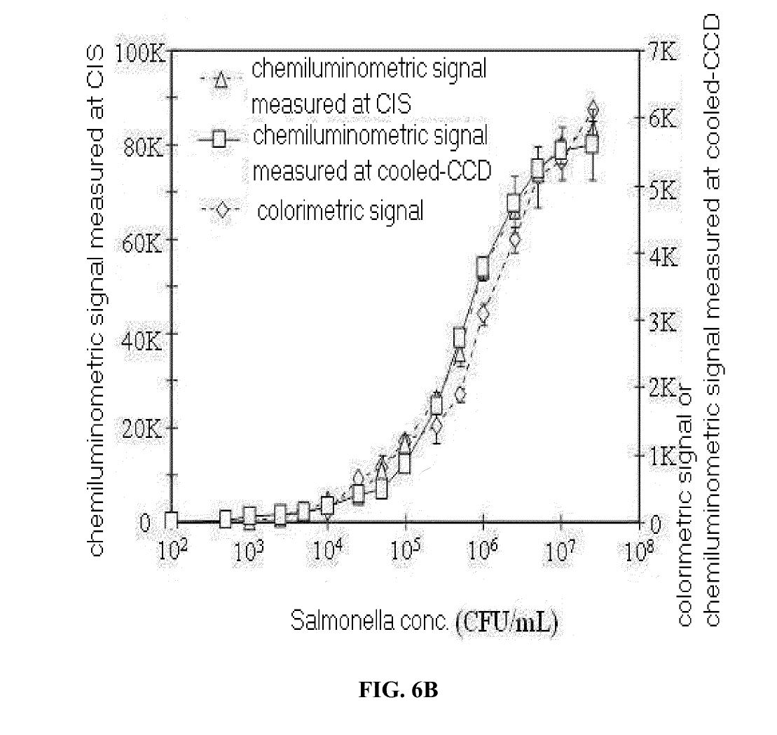

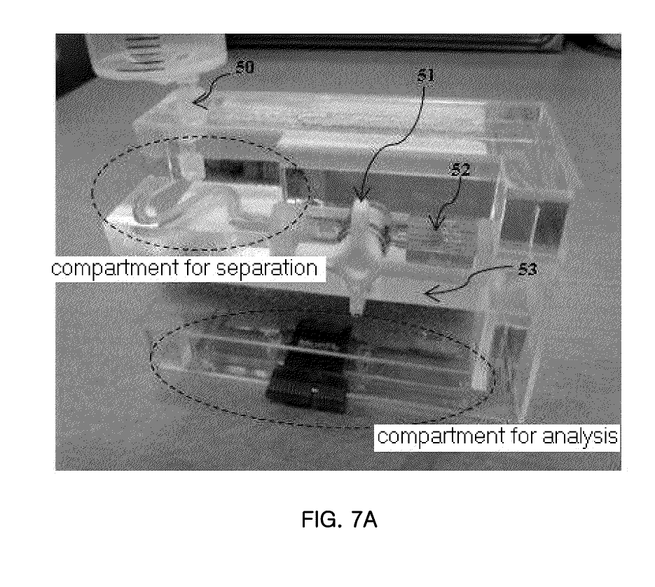

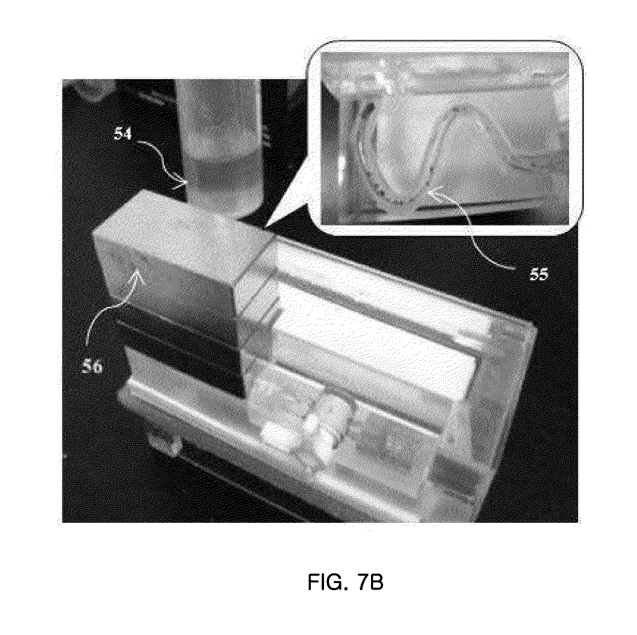

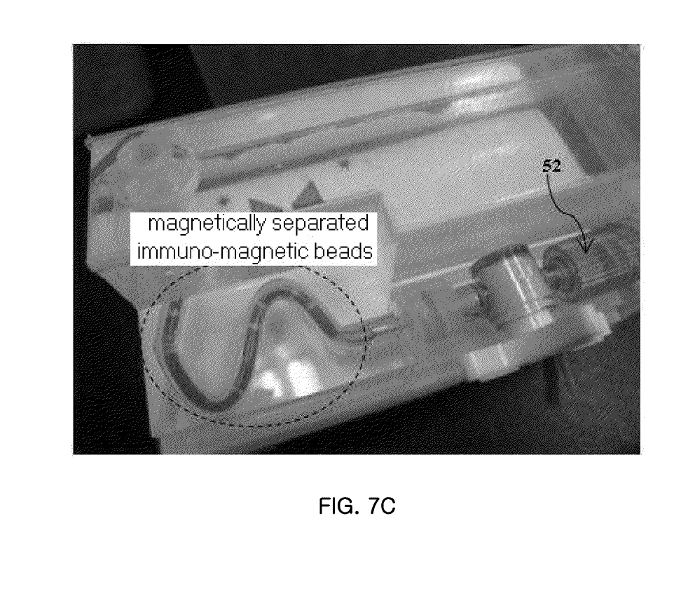

Upon observing the analytical performances of the signal detection sensors, i.e., CIS, cooled-CCD, and a web camera, the portable biosensor of the present invention measured by CIS showed a sensitivity of 4.22×10³CFU/mL, and the cooled-CCD showed a measured sensitivity of 2.54×10³ CFU/mL, which was about 1.7 times higher than that of CIS. Additionally, the colorimetric signal using the web camera showed a sensitivity of 4.75×10³ CFU/mL, similar to that of CIS (FIGS. 5, 6A, and 6B).

The sensitivity was highest when measured by cooled-CCD, which, however, a cooled-CCD cannot be portable because of its bulky size due to a cooling system installed therein, and, thus, is not suitable for the purpose of the present invention. In contrast, CIS was low in its light capture efficiency due to the absence of a cooling system. However, CIS could increase its detection sensitivity by minimizing the loss of the light signal by putting the signal generation pad 33c of the fluidic channel and CIS in close contact, thereby maximizing the photoelectron transfer efficiency. In case of the colorimetric signal, it cannot promptly perform measurements because a reaction cannot be initiated until the colorimetric signal become saturated by the accumulation of color development after the addition of an enzyme substrate solution, and, thus, is more suitable for an analytical method not requiring a signal detection sensor.

### Detection of Salmonella Using a Magnetic Enrichment Module and a Portable Biosensor

### Example 2

### (1) Manufacture of Magnetic Enrichment Module

A magnetic enrichment module consisted of a compartment for magnetic separation, a compartment for absorbing a sample solution, and a compartment for analyzing a target analyte. The main body around the device, made of plastic and an acrylic material, is in a hexagonal shape (45×100×65 mm), and was designed to have a 3-layered functional configuration, arranged in the order of a compartment for analysis based on EOC, a compartment for sample absorption, and a compartment for separation of magnetism from the bottom. For magnetic separation, Tygon® tubing (inner radius: 2 mm, thickness: 1 mm; Saint-Gobain, France) was disposed in a rectangular channel (radius: 4 mm) patterned in the shape of a sine curve (20×40 mm) and fixed thereon. Additionally, recycling thereof was made easy by preparing replaceable tubing. Furthermore, a magnet was disposed on top of the corresponding plane so that magnetic beads could be captured while moved through the tubing. Both ends of the tubing were protruded externally from the acryl and connected toward the absorption pad and the analytical device respectively. The inlet and outlet were manually controlled by the 3-way stopcock. The aqueous medium was absorbed easily by installing a support such that a cellulose membrane bundle could be in contact with one end of the lid.

### (2) Coupling of Magnetic Beads to Antibodies

Magnetic beads (33.5 μL in 30 mg/mL) were suspended in PBS (1 mL) and then the magnetic beads were separated using a magnet. The separated magnetic beads were reacted in PBS (14 μL) containing 20 μg of anti-Salmonella antibodies. Then, 3 M ammonium sulfate (10 μL) was added thereto and allowed to react at 37° C. for 18 hours. The remaining unreacted solution was separated out using a magnet, and the surface of the magnetic beads were blocked with 0.5% casein in PBS (casein-PBS) at 37° C. for 1 hour. After separation in the same manner, the magnetic beads-antibody conjugate was suspended in PBS (0.1 mL; 10 mg/mL magnetic beads) and then stored at 4° C.

### (3) Magnetic Enrichment of Sample

In order to concentrate the Salmonella sample, a culture medium (10 mL) was added to the magnetic beads-antibody conjugates (100 μL) and allowed to react for 15 minutes. The mixture was transferred to a syringe and then connected to a sample inlet port of a magnetic enrichment module, and the solution was directed thereinto by gravity. The magnetic beads were separated under the magnetic field, and the aqueous medium was allowed to be absorbed into the medium. Upon completion of the procedure, 0.1 M glycine buffer, pH 1.7, (54 μL) was supplied to the tubing, allowed to react for 10 minutes, and then the bacteria cells were dissociated from the magnetic beads. The bacteria cells dissociated from the magnetic beads were directed into the sample inlet port adjusting the 3-way stopcock, neutralized by mixing with 1 M Tris HCl, pH 8.5, (46 μL), and analyzed on the EOC biosensor.

### Experimental Example 3

### Measurement of Magnetic Enrichment Yield

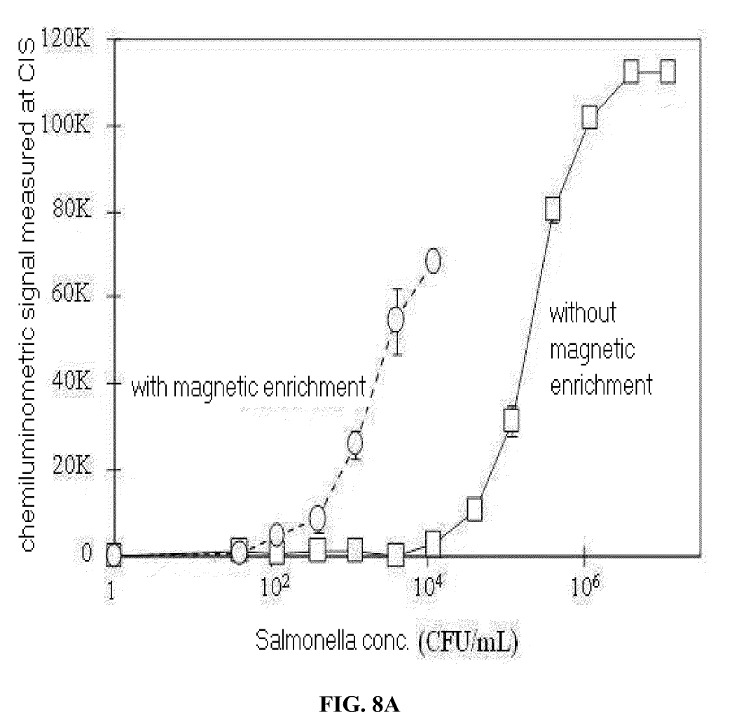

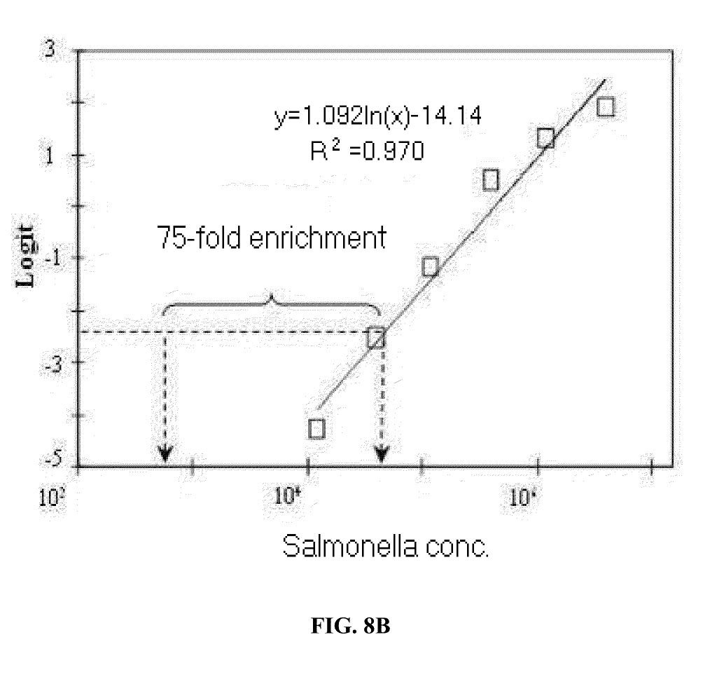

In this experiment, the enrichment yield of bacterial cells after magnetic separation was determined. The CIS light signals corresponding to each Salmonella concentration were measured, and a dose-response curve was obtained therefrom (FIG. 8A). The dose-response curve was converted to a regression line via logit-log transformation (FIG. 8B), and the regression line showed a high correlation (correlation coefficient; R²=0.970).

In order to determine the magnetic enrichment yield, a sample containing a low Salmonella concentration of 4.0×10² CFU/mL was enriched using the magnetic enrichment module, and the enriched sample was analyzed by the portable biosensor of the present invention. The obtained signal was extrapolated on the regression line and the result confirmed that Salmonella concentration in the sample was 3.0×10⁴ CFU/mL.

Consequently, it was confirmed that a 75-fold enrichment of the sample was achieved by the magnetic enrichment.

### Experimental Example 4

### Measurement Sensitivity According to Magnetic Enrichment

The light signal for the sample enriched by magnetic enrichment was measured by the portable biosensor of the present invention. Salmonella concentration in the sample was varied, and the light signals for the respective Salmonella concentrations were measured, which were graphed as the dose-response curve (FIG. 8A).

Upon comparing the sensitivities of the light signals for the samples measured with and without magnetic enrichment, the sample treated with magnetic enrichment showed a sensitivity of 1.1×10² CFU/mL whereas the sample not treated with magnetic enrichment showed a sensitivity of 7.2×10³ CFU/mL. Accordingly, it was confirmed that detection limit was improved about 67-fold when the sample underwent magnetic enrichment.

### Detection of Salmonella in Real Sample Using the Portable Biosensor

### Example 3

Flat fish purchased from a local market was used as real sample. Considering that even a trace amount of Salmonella was not allowed in foods because it was a food-borne pathogen, experiments were performed by simulating the experimental conditions as if the real samples used were contaminated with a trace amount of Salmonella.

Fish muscle in the amount of 10 g was mixed with 90 mL of a selective medium and then ground for 10 minutes. The sample was inoculated with 3.3 CFU of Salmonella, and then cultured in a continuously shaking incubator maintained at 37° C.

### Experimental Example 5

### Detection of Salmonella in Real Sample with or without Magnetic Enrichment

Real samples were collected at a 3-hour interval from the culture medium used in Example 3 and the immunity of Salmonella was measured using the portable biosensor of the present invention. Additionally, a culture medium was also collected in the same manner except for the subjection to magnetic enrichment, and an immunoassay regarding Salmonella was performed using the portable biosensor of the present invention.

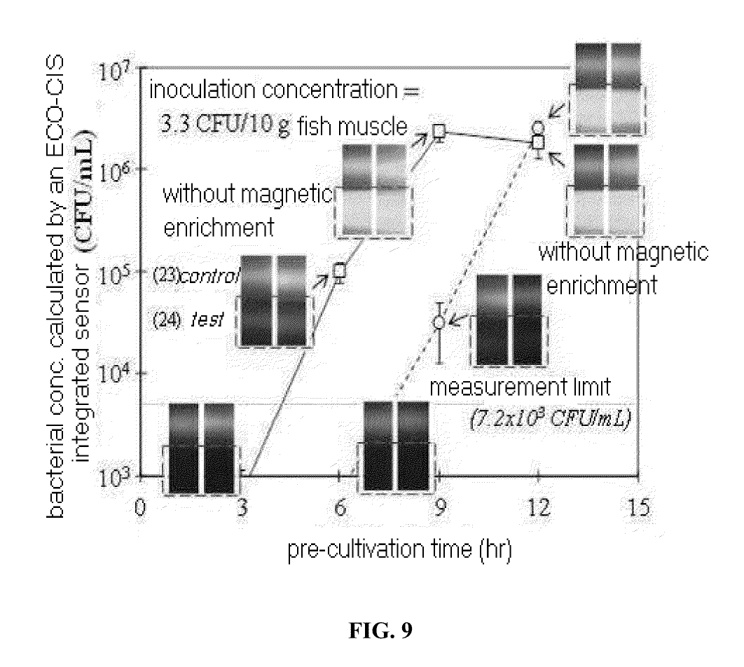

In the sample subjected to magnetic enrichment, Salmonella was detected at a time point passing 6 hours after the onset of the Salmonella inoculation, whereas in the sample without magnetic enrichment, Salmonella was detected at a time point passing 9 hours after the onset of the Salmonella inoculation, which was 3 hours later than the measurement with magnetic enrichment (FIG. 9).

Accordingly, the magnetic enrichment enabled to detect the presence of bacterium within a relatively short period of time, and thus enabled a prompt determination whether or not to distribute the foods.

As described hitherto, the portable biosensor of the present invention has the advantage that it can be miniaturized to be pocket-sized by placing a lens-free CIS as a signal detection sensor in the proximity of a light signal-generating area in the fluidic channel of EOC.

Additionally, the time required for detecting a light signal was very short (around 30 seconds) after the supply of the enzyme substrate solution, thus enabling a fast measurement and the biosensor of the present invention was also inexpensive.

As described above, optimal embodiments of the present invention have been disclosed in the drawings and the specification. Although specific terms have been used in the present specification, these are merely intended to describe the present invention and are not intended to limit the meanings thereof or the scope of the present invention described in the accompanying claims. Therefore, those skilled in the art will appreciate that various modifications and other equivalent embodiments are possible from the embodiments. Therefore, the technical scope of the present invention should be defined by the technical spirit of the claims.

What is claimed is:

1. A portable biosensor, comprising a fluidic channel, wherein the fluidic channel includes a porous solid matrix on which a capture recognition material of an analyte is fixed, and is in contact with a signal detection sensor, said signal detection sensor being a lens-free complementary metal oxide-semiconductor (CMOS) image sensors (CISs) for measuring a light signal generated by the reaction between the analyte and the capture recognition material.

2. The portable biosensor of claim 1, wherein the capture recognition material is at least one selected from the group consisting of an antibody reacting with an analyte or a conjugated analyte, an enzyme, a receptor, hexane, an aptamer, a peptide, and a molecular-imprinted artificial membrane.

3. The portable biosensor of claim 1, wherein the fluidic channel is arranged in the order of a sample application pad, a conjugate pad, a signal generation pad, and a sample absorption pad; the neighboring pads are partially overlapped, the analyte is introduced vertically along the fluidic channel while a substrate solution is introduced horizontally relative to the fluidic channel.

4. The portable biosensor of claim 3, wherein the signal generation pad consists of control lines and analytical lines

5. The portable biosensor of claim 4, wherein the lens-free complementary metal oxide-semiconductor (CMOS) image sensors (CISs) are located on the control line and the analytical line of the signal generation pad, respectively.

6. The portable biosensor of claim 1, wherein the lens-free complementary metal oxide-semiconductor (CMOS) image sensors (CISs) are fixed on a printed circuit board.

7. The portable biosensor of claim 1, wherein the light signal is luminescent, fluorescent, and colorimetric, wherein the signal detection sensor used for measuring the light signal includes a lens-free charge coupled device (CCD), a photodiode, or a photomultiplier tube in addition to CIS.

8. The portable biosensor of claim 1, wherein the light signal is converted into an electric signal using a signal detection device, wherein an analog-to-digital converter, a central processing unit, a synchronous random access memory, a volatile memory, a secure digital card, and a liquid crystal display unit are assembled thereinto, wherein the volatile memory is NAND or NOR.

9. A method for detecting an analyte, comprising:
a) loading an analyte into a sample application port of a fluidic channel comprising a porous solid matrix, on which a capture recognition material of an analyte is fixed;
b) generating a light signal by the reaction between the analyte and the capture recognition material in the fluidic channel; and
c) detecting the analyte by measuring the light signal with the portable biosensor of claim 1.

10. The method of claim 9, further comprising performing a magnetic enrichment of a sample comprising the analyte using magnetic beads to which the capture recognition material of the analyte is fixed, prior to performing step a).
